(19) United States
(12) Patent Application Publication
(10) Pub. No.: US 2010/0104649 A1
(43) Pub. Date: Apr. 29, 2010

(54) Lercanidipine Hydrochloride Polymorphs and an Improved Process for Preparation of 1,1,N-Trimethyl-N-(3,3-Diphenylpropyl)-2-Aminoethyl Acetoacetate

(75) Inventors: Girish Dixit, Bangalore (IN); Krishnadatt Baldevprasad Sharma, Navi-Mumbai (IN); Nitin Sharadchandra Pradhan, Maharashtra (IN); Jon Valgeirsson, Hafnarfjordur (IS)

Correspondence Address: SALIWANCHIK LLOYD & SALIWANCHIK;A PROFESSIONAL ASSOCIATION, PO Box 142950, GAINESVILLE, FL, 32614, US

(73) Assignee: ACTAVIS GROUP PTC EHF, Hafnarfjordur (IS)

(21) Appl. No.: 12/530,101

(22) PCT Filed: Mar. 5, 2008
(86) PCT No.: PCT/IB2008/000903

§ 371 Date: Jan. 12, 2010

(30) Foreign Application Priority Data
Mar. 5, 2007 (IN) 434/CHE/2007
Mar. 12, 2007 (IN) 498/CHE/2007
Apr. 5, 2007 (IN) 720/CHE/2007

(51) Int. Cl. A61K 9/14; C07D 213/80; C07C 69/66; A61K 31/44; A61P 9/12

(52) U.S. Cl. 424/489; 546/321; 560/174; 514/356

(57) ABSTRACT

Disclosed herein is an improved, commercially viable and industrially advantageous process for the preparation of substantially pure Lercanidipine intermediate, 1,1,N-trimethyl-N-(3,3-diphenylpropyl)-2-aminoethyl acetoacetate. The intermediate is useful for preparing Lercanidipine, or a pharmaceutically acceptable salt thereof, in high yield and purity. The present invention further provides a novel crystalline form of Lercanidipine hydrochloride and a process for its preparation. The present invention also provides a process for the preparation of amorphous form of Lercanidipine hydrochloride.

### CROSS-REFERENCE TO RELATED APPLICATIONS

This application claims the benefit of priority to Indian provisional application Nos. 434/CHE/2007, filed on Mar. 5, 2007, 498/CHE/2007, filed on Mar. 12, 2007, as well as 720/CHE/2007, filed on Apr. 5, 2007 which are incorporated herein by reference.

### FIELD OF THE INVENTION

Disclosed herein is an improved, commercially viable and industrially advantageous process for the preparation of substantially pure Lercanidipine intermediate, 1,1,N-trimethyl-N-(3,3-diphenylpropyl)-2-aminoethyl acetoacetate. The intermediate is useful for preparing Lercanidipine, or a pharmaceutically acceptable salt thereof, in high yield and purity. The present invention further provides a novel crystalline form of Lercanidipine hydrochloride and a process for its preparation. The present invention also provides a process for the preparation of amorphous form of Lercanidipine hydrochloride.

### BACKGROUND OF THE INVENTION

U.S. Pat. No. 4,705,797 discloses a variety of 1,4-dihydro-2,6-dimethyl-pyridine-3,5-dicarboxylic acid derivatives, and their stereoisomers and salts, process for their preparation, pharmaceutical compositions comprising the derivatives, and method of use thereof. These compounds are antihypertensive agents. Among them, Lercanidipine, 1,4-dihydro-2,6-dimethyl-4-(3-nitrophenyl)-3,5-pyridinedicarboxylic acid 2-[(3,3-diphenylpropyl)methylamino]-1,1-dimethylethyl methyl ester, is a highly lipophilic dihydropyridine calcium antagonist with a long duration of action and high vascular selectivity. Lercanidipine is useful as an anti-hypertensive. Lercanidipine lowers blood pressure by blocking calcium channels of arterial smooth muscle, thus decreasing peripheral vascular resistance. Lercanidipine is represented by the following structural formula:

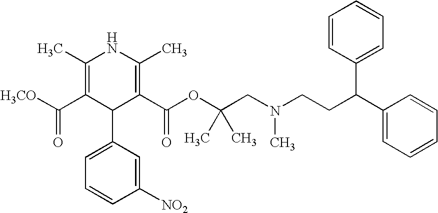

The hydrochloride salt of Lercanidipine is a useful antihypertensive calcium channel blocker sold under the brand names Lercadip, Lerdip, Lerzam, Zanedip, and Zanidip®. Methods of preparing Lercanidipine hydrochloride, as well as methods of resolving Lercanidipine into individual enantiomers are described in U.S. Pat. No. 4,705,797, U.S. Pat. No. 4,968,832, U.S. Pat. No. 5,767,136, U.S. Pat. No. 5,912,351, U.S. Pat. No. 5,696,139, and U.S. Patent Application Nos. US 2003/0069285 and US 2003/0083355.

In the preparation of Lercanidipine, 1,1,N-trimethyl-N-(3,3-diphenylpropyl)-2-aminoethyl acetoacetate of formula I:

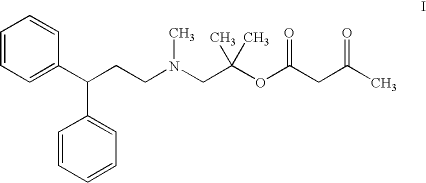

is a key intermediate. According to U.S. Pat. No. 4,705,797 (hereinafter referred to as the '797 patent), Lercanidipine hydrochloride is prepared by the reaction of 2,N-dimethyl-N-(3,3-diphenylpropyl)-1-amino-2-propanol with diketene to produce 1,1,N-trimethyl-N-(3,3-diphenylpropyl)-2-aminoethyl acetoacetate of formula I, which is then coupled with 3-nitrobenzaldehyde to produce 1,1,N-trimethyl-N-(3,3-diphenylpropyl)-2-aminoethyl α-acetyl-3-nitrocinnamate followed by cyclization with methyl 3-aminocrotonate in isopropanol at reflux temperature. Lercanidipine is isolated as its hydrochloride by crystallization from water containing HCl and NaCl.

The '797 patent involves the use of diketene for the preparation of 1,1,N-trimethyl-N-(3,3-diphenylpropyl)-2-aminoethyl acetoacetate. This process suffers from drawbacks since diketene is an explosive and hazardous chemical and use of diketene is not advisable for scale up operations. Moreover, Lercanidipine hydrochloride obtained by the processes described in the '797 patent does not have satisfactory purity. Unacceptable amounts of impurities are formed during the reaction between 2,N-dimethyl-N-(3,3-diphenylpropyl)-1-amino-2-propanol and diketene, thus resulting in a poor product yield. The process also involves column chromatographic purifications. Methods involving column chromatographic purifications are generally undesirable for large-scale operations, thereby making the process commercially unfeasible.

A need remains for an improved and commercially viable process of preparing a substantially pure 1,1,N-trimethyl-N-(3,3-diphenylpropyl)-2-aminoethyl acetoacetate of formula Ito resolve the problems associated with the processes described in the prior art. Desirable process properties include non-hazardous and environmentally friendly, easy to handle reagents, reduced cost, and greater simplicity and suitable for large-scale preparation, increased purity and increased yield of the product, thereby enabling the production of Lercanidipine and its pharmaceutically acceptable acid addition salts in high purity and in high yield.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products. Polymorphic forms of a compound can be distinguished in the laboratory by analytical methods such as X-ray diffraction (XRD), Differential Scanning Calorimetry (DSC) and infrared spectrometry (IR).

Solvent medium and mode of crystallization play very important role in obtaining a crystalline form over the other.

Lercanidipine hydrochloride can exist in different polymorphic forms, which differ from each other in terms of stability, physical properties, spectral data and methods of preparation.

U.S. Pat. No. 5,912,351 discloses process for the preparation of Lercanidipine hydrochloride by reaction of 2,6-dimethyl-5-methoxycarbonyl-4-(3-nitrophenyl)-1,4-dihydropyridine-3-carboxylic acid with thionyl chloride in dichloromethane and dimethylformamide and subsequent esterification of the obtained acid chloride with 2,N-dimethyl-N-(3,3-diphenylpropyl)-1-amino-2-propanol. The process yields Lercanidipine hydrochloride in an anhydrous non-hygroscopic crystalline form. The product is purified by crystallization to give Lercanidipine hydrochloride having a melting point of 186-188° C., later designated Form C.

U.S. Pat. No. 6,852,737 (hereinafter referred to as the '737 patent) discloses crystalline Forms I and II and crude Forms A and B of Lercanidipine hydrochloride and processes for their preparation. Crude Form A of Lercanidipine hydrochloride is described in Example 2 as having a differential scanning calorimetric (DSC) peak of 150-152° C. Crude Form B of Lercanidipine hydrochloride is described in Example 3 as having a DSC peak of 131-135° C. Additionally, the '737 patent discloses that thermogravimetric studies show that crude Form A contains 3-4% residual ethyl acetate, crude Form B contains 0.3-0.7% residual ethyl acetate, and crude Form C contains 0-0.1% residual solvents. Crystalline Forms I and II of lercanidipine hydrochloride are well characterized by XRD analysis in the '737 patent. Example 12 of the '737 patent discloses that crystalline Form I has a melting temperature (T peak) of 198.7° C. and an onset temperature of 179.8° C., and crystalline Form II has a melting temperature (T peak) of 209.3° C. and an onset temperature of 169° C.

PCT publication No. WO 2003/014085 describes the formation of solvates of Lercanidipine hydrochloride with various organic solvents, and crystalline forms (III) and (IV) which are obtainable from the solvates by removing the solvation solvent. The solvates and crystalline forms Lercanidipine hydrochloride are prepared by using Forms (I), (A) or (B) as starting material.

PCT Publication WO 2006/089787 discloses amorphous Lercanidipine hydrochloride having a purity of at least 95% and a method for its preparation. The method includes dissolving crystalline Lercanidipine hydrochloride in an organic solvent to provide a solution and isolating amorphous Lercanidipine hydrochloride either by (a) adding water to the solution to form a precipitate and collecting the precipitate or (b) evaporating off the organic solvent.

PCT publication No. WO 2007/031865 discloses novel crystalline Form V of Lercanidipine hydrochloride and use thereof for the preparation of amorphous Lercanidipine hydrochloride.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic. A new polymorphic form of Lercanidipine hydrochloride and process for the preparation of substantially pure amorphous Lercanidipine hydrochloride now has been discovered.

Accordingly, there remains a need for novel polymorph of Lercanidipine hydrochloride and also an improved process for preparing substantially pure amorphous Lercanidipine hydrochloride.

### SUMMARY OF THE INVENTION

The present inventors have surprisingly found that Lercanidipine intermediate, 1,1,N-trimethyl-N-(3,3-diphenylpropyl)-2-aminoethyl acetoacetate, can be prepared in high purity and with high yield by reacting 2,N-dimethyl-N-(3,3-diphenylpropyl)-1-amino-2-propanol with a protected acetoacetic acid compound in the presence of a metal catalyst.

The present invention provides an efficient, convenient, commercially viable and environment friendly process for the preparation of Lercanidipine intermediate, 1,1,N-trimethyl-N-(3,3-diphenylpropyl)-2-aminoethyl acetoacetate in an 80-90% overall yield. Advantageously, the reagents used for present invention are less hazardous and easy to handle at commercial scale and also involves less expensive reagents.

We have now surprisingly and unexpectedly discovered a novel crystalline form of Lercanidipine hydrochloride with adequate stability and good dissolution properties, designated as Lercanidipine hydrochloride Form Y, characterized by an X-ray powder diffraction pattern having peaks expressed as 2θ angle positions at about 4.7 and 5.0+0.2 degrees.

In one aspect, the present invention provides a novel and stable crystalline form Y of Lercanidipine hydrochloride and use thereof for the preparation of amorphous Lercanidipine hydrochloride.

In another aspect, the present invention further encompasses a process for preparing the highly pure and stable crystalline form Y of Lercanidipine hydrochloride.

In another aspect, the present invention further encompasses a process for preparing the highly pure and stable amorphous form of Lercanidipine hydrochloride.

In another aspect, the present invention provides a pharmaceutical composition comprising the novel crystalline Lercanidipine hydrochloride Form Y of the present invention and one or more pharmaceutically acceptable excipients.

In still another aspect, the present invention provides a pharmaceutical composition comprising the novel crystalline Lercanidipine hydrochloride Form Y made by the process of the present invention, and one or more pharmaceutically acceptable excipients.

In still further aspect, the present invention further encompasses a process for preparing a pharmaceutical formulation comprising combining the novel crystalline Lercanidipine hydrochloride Form Y of the present invention with one or more pharmaceutically acceptable excipients.

### BRIEF DESCRIPTION OF THE DRAWING

FIG. 1 is a characteristic powder X-ray diffraction (XRD) pattern of crystalline Lercanidipine hydrochloride Form Y.

FIG. 2 is a characteristic differential scanning calorimetric (DSC) thermogram of crystalline Lercanidipine hydrochloride Form Y.

FIG. 3 is a characteristic thermogravimetric analysis (TGA) of crystalline Lercanidipine hydrochloride Form Y.

FIG. 4 is a characteristic infra red (IR) spectrum of crystalline Lercanidipine hydrochloride Form Y.

FIG. 5 is a characteristic powder X-ray diffraction (XRD) pattern amorphous form of Lercanidipine hydrochloride.

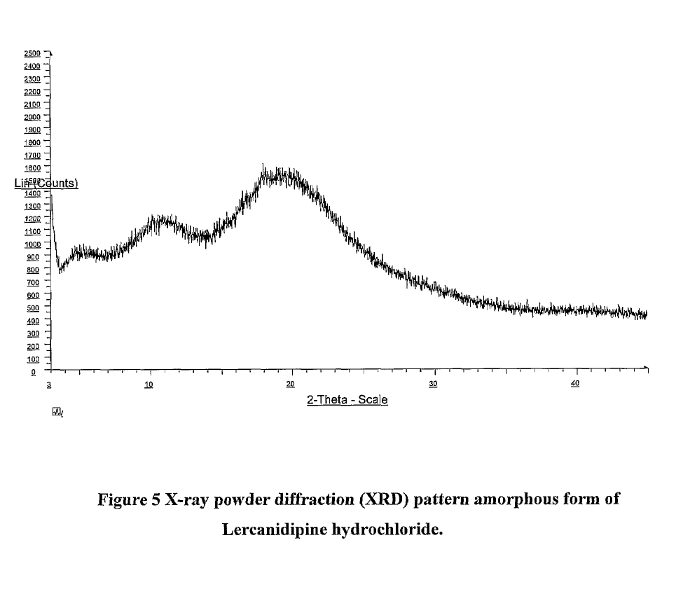

The X-Ray powder diffraction was measured by an X-ray powder Diffractometer equipped with CuKα-radiations (40 kV, 40 mA) in wide—angle X-ray Diffractometer of BRUKER axs, D8 ADVANCE. The sample was analyzed using the following instrument parameters: measuring range=3-45° 2-theta; step width=0.01579°; and measuring time per step=0.11 sec.

DSC (Differential Scanning Calorimetry) measurements were performed with a Differential Scanning Calorimeter (DSC Q1000, TA Instruments, New Castle, Del., USA) at a scan rate of 10° C. per minute The nitrogen gas purge at 50 ml/min. The instrument was calibrated for temperature and heat flow using indium as standards. The samples were encapsulated in to closed aluminium pans subsequently crimped to ensure a tight seal. Data acquisition and analysis were performed using Universal analysis 2000 software (TA Instruments).

Thergravimetric analysis was performed with a TGA Q500 of TA instruments, Lukens Drive, Del., USA. The gradual weight loss has been observed from 160° C.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 4000 to 450 cm⁻¹.

### DETAILED DESCRIPTION OF THE INVENTION

Unless otherwise indicated, the following definitions are set forth to illustrate and define the meaning and scope of the various terms used to describe the invention herein.

The term “crystalline polymorph” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of Lercanidipine hydrochloride in accordance with the present invention preferably contains less than about 10 percent crystalline forms of Lercanidipine hydrochloride, more preferably less than 5 percent crystalline form of Lercanidipine hydrochloride, and still more preferably is essentially free of crystalline forms of Lercanidipine hydrochloride. “Essentially free of crystalline forms of Lercanidipine hydrochloride” means that no crystalline polymorph forms of Lercanidipine hydrochloride can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

According to one aspect of the present invention, there is provided a process for the preparation of the Lercanidipine intermediate, 1,1,N-trimethyl-N-(3,3-diphenylpropyl)-2-aminoethyl acetoacetate of formula I:

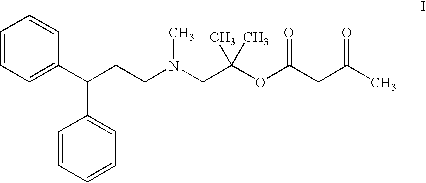

which comprises:
reacting 2,N-dimethyl-N-(3,3-diphenylpropyl)-1-amino-2-propanol of formula II:

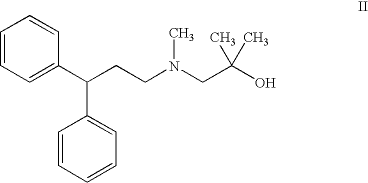

with a protected acetoacetic acid compound of formula III:

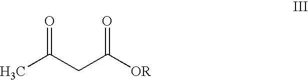

wherein R is an alkyl or an aryl group, in the presence of a metal catalyst in a suitable solvent to give substantially pure 1,1,N-trimethyl-N-(3,3-diphenylpropyl)-2-aminoethyl acetoacetate of formula I.

Exemplary metal catalysts include, but are not limited to, Zn, Sc, Ti, V, Cr, Mn, Fe, Co, Ni and Cu. Most preferable metal catalyst is Zn.

Exemplary solvents include, but are not limited to, non-polar solvents, aprotic solvents, alcohol solvents, and mixtures thereof. Exemplary non-polar solvents include, but are not limited to, toluene, xylene, and mixtures thereof. Exemplary aprotic solvents include, but are not limited to, dimethylformamide, dimethylacetamide, dimethylsulfoxide, and mixtures thereof. Exemplary alcohol solvents include, but are not limited to, aromatic and aliphatic C₁-C₁₂ alcohols solvents, and the like, and mixtures thereof. Exemplary aliphatic alcohol solvents include, but are not limited to, methanol, ethanol, n-propanol, isopropanol, n-butanol, isobutanol, tert-butanol, and mixtures thereof. Exemplary aromatic alcohol solvents include, but are not limited to, benzyl alcohol, benzyloxyethanol, phenoxyethanol and the like, and mixtures thereof. Specific solvents are toluene, xylene, dimethylformamide, and mixtures thereof, and more specifically toluene and xylene.

The lower alcohols such as methanol, ethanol, isopropanol etc. generated in the reaction required to remove in order to precede the reaction towards completion.

Specifically, the protecting group R includes, but are not limited to, methyl, ethyl, n-propyl, isopropyl, n-butyl, t-butyl, pentyl, and benzyl groups, and more specifically methyl.

The reaction is carried out at a temperature of 40° C. to the reflux temperature of the solvent used, specifically at a temperature of 50° C. to the reflux temperature of the solvent used, more specifically at a temperature of 80° C. to the reflux temperature of the solvent used, and most specifically at the reflux temperature of the solvent used.

As used herein, “reflux temperature” means the temperature at which the solvent or solvent system refluxes or boils at atmospheric pressure.

Usually, about 0.5 to 6 moles, specifically, about 2 to 4 moles of metal catalyst is used per 1 mole of 2,N-dimethyl-N-(3,3-diphenylpropyl)-1-amino-2-propanol.

Usually, about 3 to 15 moles, specifically, about 8 to 11 moles of the protected acetoacetic acid compound of formula III is used per 1 mole of 2,N-dimethyl-N-(3,3-diphenylpropyl)-1-amino-2-propanol.

The process of the present invention allows the product to be easily isolated and purified, thereby producing a product with 80-90% overall yield.

The compounds of formula I obtained by the process disclosed herein, have a purity (measured by High Performance Liquid Chromatography, hereinafter referred to as ‘HPLC’) greater than about 98%, specifically greater than about 99%, and more specifically greater than about 99.5%.

The use of inexpensive, non-explosive, non-hazardous, readily available and easy to handle reagents allows the process disclosed herein to be suitable for preparation of Lercanidipine at lab scale and in commercial scale operations.

In one embodiment, the compound of formula I obtained is isolated from a suitable organic solvent by methods usually known in the art such as cooling, partial removal of the solvent from the solution, addition of precipitating solvent or a combination thereof.

The term “substantially pure compound of formula I refers to the compound of formula I having purity greater than about 98%, specifically greater than about 99%, and more specifically greater than about 99.5% measured by HPLC.

Lercanidipine and pharmaceutically acceptable salts of Lercanidipine can be prepared in high purity by using the substantially pure compound of formula I obtained by the methods disclosed herein, by known methods, for example as described in U.S. Pat. No. 4,705,797.

According to another aspect of the present invention, there is provided a novel crystalline form of Lercanidipine hydrochloride, designated as Lercanidipine hydrochloride Form Y, characterized by an X-ray powder diffraction pattern having peaks expressed as 2-theta angle positions at about 4.7 and 5.0±0.2 degrees.

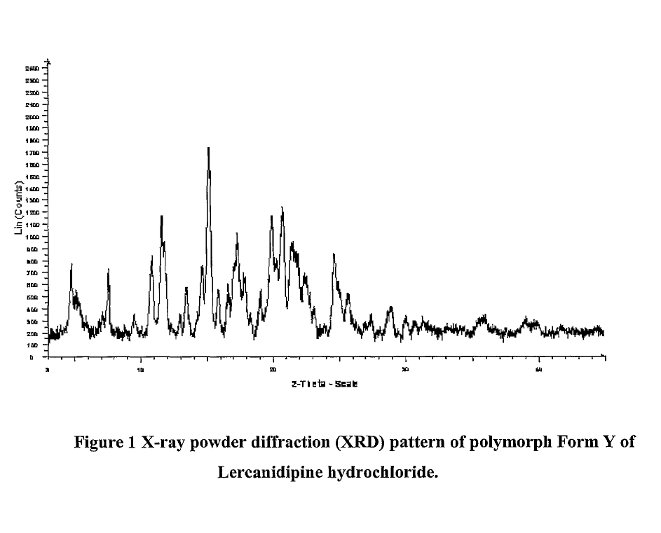

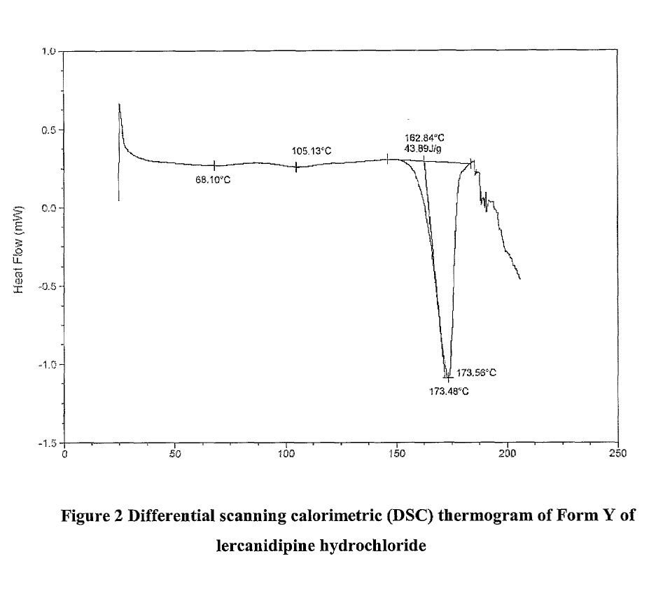

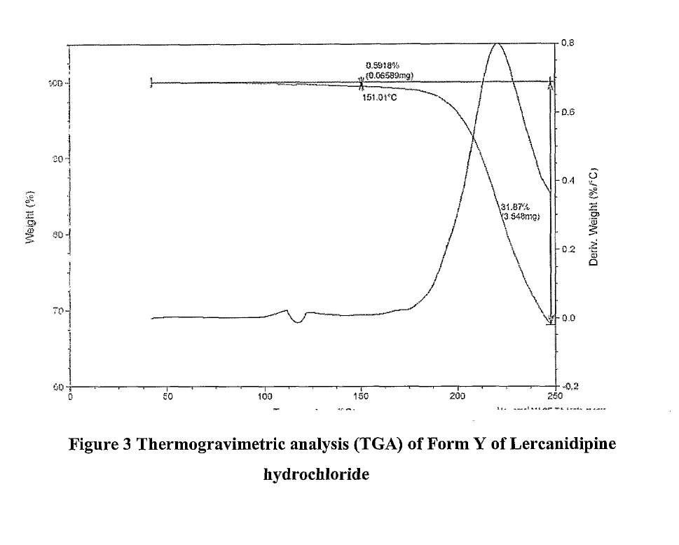

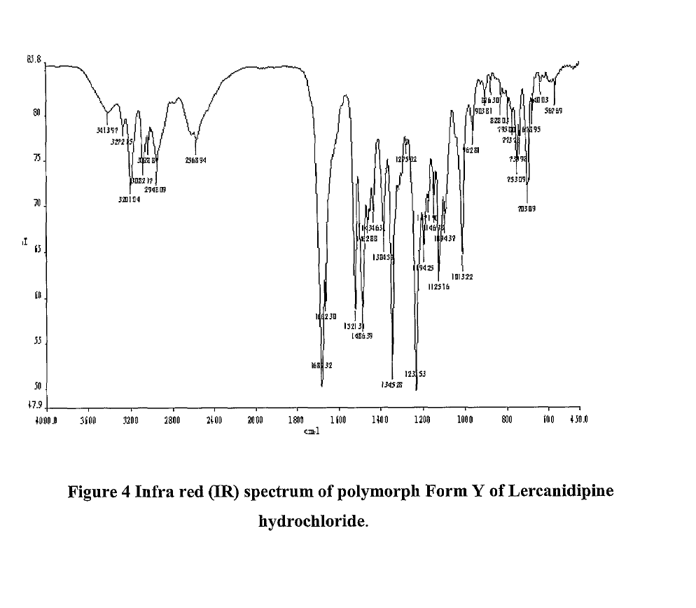

Lercanidipine hydrochloride Form Y may be further characterized by at least one of the following: a powder XRD pattern having additional peaks at about 7.0, 18.9, 23.1 and 24.9±0.2 degrees 2-theta substantially as depicted in FIG. 1; a DSC thermogram having an endotherm peak at about 173° C. substantially as depicted in FIG. 2; a TGA thermogram substantially in accordance with FIG. 3; and/or an IR spectrum substantially in accordance with FIG. 4.

According to another aspect of the present invention, a process for the preparation of crystalline Lercanidipine hydrochloride Form Y is provided, which comprises:

a) providing a solution of Lercanidipine hydrochloride in an amide solvent;
b) adding an aliphatic ester solvent to the solution; and
c) recovering substantially pure Lercanidipine hydrochloride Form Y from the solution.

Exemplary amide solvents include, but are not limited to, N,N-dimethylacetamide, N,N-diethylacetamide, N,N-dimethylacetoacetamide, N,N-diethylacetoacetamide, formanilide, N-methyl formanilide, N,N-di-n-propyl acetamide, N,N-di-isopropyl acetamide, Di-n-butyl-acetamide, N,N-dimethyl-2,2-diphenyl acetamide, and mixtures thereof. More specific amide solvent is N,N-dimethylacetamide.

Step-(a) of providing a solution of Lercanidipine hydrochloride includes dissolving any form of Lercanidipine hydrochloride in a suitable amide solvent, or obtaining an existing solution from a previous processing step.

The solution instep-(a) may also be prepared by admixing Lercanidipine free base, hydrochloric acid and the amide solvent to obtain a mixture; and heating the mixture to obtain a Lercanidipine hydrochloride solution. Heating the mixture to obtain a Lercanidipine hydrochloride solution is preferably to a temperature of about 20° C. to about 100° C., more preferably of about 50° C. to about 100° C.

Exemplary aliphatic esters used in step-(b) include, but are not limited to, ester groups having from about 2 to about 12 carbon atoms, e.g. ethyl acetate, isopropyl acetate and the like, and mixtures thereof. The aliphatic ester solvent may be present in a ratio of about 10-15 v/w with respect to the Lercanidipine hydrochloride.

The addition of aliphatic ester solvent in step-(b) is carried out at a temperature of 10° C. to 100° C., specifically at a temperature of 30° C. to 90° C., more specifically at a temperature of 50° C. to 70° C.

The substantially pure Lercanidipine hydrochloride Form Y obtained in step-(c) can be recovered by crystallization.

The Lercanidipine hydrochloride Form Y obtained in step-(c) may be collected by filtration or centrifugation.

If desired, the solution can also be seeded with Lercanidipine hydrochloride. The crystallization may take place at a temperature of about 0° C. to 35° C. for about 5 hours to about 25 hours, and preferably about 16 to about 18 hours. The resulted solid is then filtered and optionally washed with the aliphatic ester solvent.

After performing the purification operation of the present invention, substantially pure Lercanidipine hydrochloride Form Y can be prepared with a degree of purity greater than or equal to about 98.5%, preferably greater than or equal to about 99.5% and more preferably greater than or equal to about 99.7%. The purity of Lercanidipine hydrochloride Form Y of the present invention may be determined by any method known in the art, e.g., high performance liquid chromatography (HPLC) analysis.

According to another aspect of the present invention, a process for the preparation of amorphous Lercanidipine hydrochloride is provided, which comprises:

a) suspending Lercanidipine hydrochloride in water;
b) heating the suspension; and
c) recovering Lercanidipine hydrochloride in amorphous form.

In step (b), the suspension of Lercanidipine hydrochloride is heated to a temperature ranging from about 60° C. to about 100° C., preferably at about 95° C. to 100° C. for a time period ranging from about 40 minutes to about 120 minutes. The suspension can then be stirred at 100° C. for a time period ranging from about 60 minutes to about 120 minutes. The suspension can then be cooled to temperature ranging from about 5° C. to about 10° C. and further stirred for a time period ranging from about 40 minutes to about 90 minutes.

In step (c) of the process of the present invention, the amorphous Lercanidipine hydrochloride can be recovered by, for example, collecting the precipitate of the amorphous Lercanidipine hydrochloride. The resulting solid then filtered and washed with water and can be dried, e.g., under vacuum (not less than about 700 mm) at a temperature ranging from about 60° C. to about 70° C. until the amorphous Lercanidipine hydrochloride meets to the residual solvents as mentioned in ICH guidelines. The amorphous Lercanidipine hydrochloride obtained by present invention having a degree of purity greater than or equal to about 99.0%, preferably greater than or equal to about 99.5.0% and more preferably greater than or equal to about 99.9%. The purity of the amorphous Lercanidipine hydrochloride of the present invention may be determined by any method known in the art, e.g., high performance liquid chromatography (HPLC) analysis.

Substantially pure Lercanidipine hydrochloride Form Y prepared by the methods disclosed herein may be formulated into pharmaceutical compositions. In one embodiment, the present invention provides a pharmaceutical composition consisting essentially of a therapeutically effective amount of substantially pure Form Y of Lercanidipine hydrochloride, and at least one component selected from the group consisting of a pharmaceutically acceptable carrier or diluent, flavorant, sweetener, preservative, dye, binder, suspending agent, dispersing agent, colorant, disintegrant, excipient, film forming agent, lubricant, plasticizer, edible oil, and a binder. In a preferred embodiment, the pharmaceutical composition or dosage form comprises about 0.1 to 400 mg substantially pure lercanidipine hydrochloride in polymorph Form Y, for all uses disclosed herein. Preferably, the composition or dosage form comprises from about 1 to 200 mg substantially pure Form Y of lercanidipine hydrochloride, more still more preferably from about 5 to 40 mg.

In one embodiment, the substantially pure Lercanidipine hydrochloride Form Y disclosed herein for use in the pharmaceutical compositions of the present invention, wherein 90 volume-% of the particles (D₉₀) have a size of less than 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure Lercanidipine hydrochloride Form Y can be achieved via comminution, or a mechanical process of reducing the size of particles which includes any one or more of cutting, chipping, crushing, milling, grinding, micronizing, trituration or other particle size reduction methods known in the art, to bring the solid state forms the desired particle size range.

Suitable pharmaceutically acceptable carriers or diluents include, but are not limited to, ethanol, water, glycerol, propylene glycol, aloe vera gel, allantoin, lactose, microcrystalline cellulose, mannitol, sodium phosphate, calcium phosphate, sugar, fructose, glucose, sorbitol, glycerin, vitamin A and E oils, mineral oil, PPG2 myristyl propionate, magnesium carbonate, potassium phosphate, vegetable oil, animal oil, and solketal.

Suitable disintegrants include, but are not limited to, starch, e.g., corn starch, sodium starch glycolate, sodium crosscarmellose, methyl cellulose, agar, bentonite, xanthan gum, sodium starch glycolate, crosspovidone and the like.

Suitable lubricants include, but are not limited to, sodium oleate, sodium stearate, sodium stearyl fumarate, magnesium stearate, stearic acid, sodium benzoate, sodium acetate, sodium chloride and the like.

A suitable film forming agent is, but is not limited to, hydroxypropyl methyl cellulose (hypromellose), ethyl cellulose, shellac, sucrose, acrylic acids derivatives (e.g. methacrylic acid copolymer, ammonio methacrylate copolymer), or mixtures of two or more of these substances, and the like.

Suitable dispersing and suspending agents include, but are not limited to, synthetic and natural gums, such as vegetable gum, tragacanth, acacia, alginate, dextran, sodium carboxymethylcellulose, methylcellulose, polyvinyl-pyrrolidone, bentonite, ethoxylated isostearyl alcohols, polyoxyethylene sorbitol and sorbitan esters, microcrystalline cellulose, aluminum metahydroxide, agar-agar and gelatin.

### HPLC Method for Measuring Chemical Purity:

Column: C18 Sun fire, 100×3.0 mm, 3.5 mu Column temperature: 50° C.
Mobile phase A: 100% Buffer [pH] solution pH3.0
Mobile phase B: 70% acetonitrile
Buffer: 4 g of sodium dihydrogenphosphate monohydrate in 1000 ml water. Adjust pH to 3.0 with concentrated phosphoric acid: UV, 210 nm,
Flow rate: 0.5 ml/min. Injection Volume: 10 [mu]
Run Time: 40 minutes
Retention time: 23.2 minutes

The following examples are given for the purpose of illustrating the present invention and should not be considered as limitation on the scope or spirit of the invention.

### EXAMPLES

### Example 1

### Preparation of 1,1,N-trimethyl-N-(3,3-diphenylpropyl)-2-aminoethyl acetoacetate

2,N-Dimethyl-N-(3,3-diphenylpropyl)-1-amino-2-propanol (5 g) and methyl acetoactate (7.5 g) were dissolved in xylene (50 ml) at 25-30° C. followed by the addition of zinc dust (1.75 g). The reaction mixture was heated at 140-145° C. for 7 hours. Next, xylene was distilled out along with simultaneous addition of xylene in order to maintain the volume of xylene in the reaction mixture. The reaction mixture was cooled at 25-30° C. followed by filtration to remove the catalyst. Distillation was carried out to remove xylene under reduced pressure. The resulting residue was degassed for 1 hour to produce title compound as viscous brown oil.

### Example 2

### Preparation of 1,1,N-trimethyl-N-(3,3-diphenylpropyl)-2-aminoethyl α-acetyl-3-nitrocinnamate hydrochloride

A mixture of 1,1, N-trimethyl-N-(3,3-diphenylpropyl)-2-aminoethyl acetoacetate (5 g) and 3-nitrobenzaldehyde (2 g) was dissolved in toluene (50 ml). The solution was cooled to 0° C. and dry hydrogen chloride gas was bubbled into it until the solution was saturated. The reaction mixture was stirred for 9 hours at 0° C. The organic layer was separated from the reaction mixture and washed with toluene (25 ml). The resulting oily residue was dissolved in methylene dichloride (100 ml). The resulting solution was dried over calcium chloride followed by the distillation of methylene dichloride under reduced pressure at 30-35° C. to produce 5.5 g of 1,1,N-trimethyl-N-(3,3-diphenylpropyl)-2-aminoethyl α-acetyl-3-nitrocinnamate hydrochloride.

### Example 3

### Preparation of 1,1,N-trimethyl-N-(3,3-diphenylpropyl)-2-aminoethyl-1,4-dihydro-2,6-dimethyl-4-(3-nitrophenyl)-pyridine-3,5-dicarboxylate Hydrochloride

1,1,N-trimethyl-N-(3,3-diphenylpropyl)-2-aminoethyl α-acetyl-3-nitrocinnamate hydrochloride (5.5 g) was dissolved in n-propanol (55 ml) at 40° C. under stirring. The resulting reaction mixture was cooled at 25-30° C. followed by the addition of triethylamine (8.2 ml) and stirred for 10 minutes. This was followed by the addition of methyl-3-aminocrotonate (2.64 g) and the resulting reaction mixture was heated at 75-80° C. for 10 hours. The reaction mixture was cooled at 25-30° C. and adjusted the pH at about 2.0 by using n-propanolic hydrochloride solution. The reaction mixture was then stirred for 30 minutes at 25-30° C. followed by the distillation of n-propanol. The resulting oily mass was dissolved in ethyl acetate (55 ml) followed by washing with water (3×25 ml). The organic layer was dried over sodium sulfate followed by the removal of ethyl acetate under reduced pressure. The resulting oily mass was dissolved in ethyl acetate (55 ml) followed by stirring at 25-30° C. for 24 hours. The yellow colored solid was precipitate out, which was filtered and washed with ethyl acetate (5 ml) and dried at 60° C. for 8 hours to produce 2.5 gm of the title compound.

### Example 4

### Preparation of Lercanidipine Hydrochloride Form Y

Lercanidipine hydrochloride (10 g) was dissolved in N,N-dimethyl acetamide (5.0 ml) at 55-60° C. under stirring. This was followed by the addition of isopropyl acetate (100 ml) under stirring at 55-60° C. The resulting solution was cooled at 25 to 30° C. followed by the addition of seeds of Lercanidipine hydrochloride. The resulting mass was stirred for 15 to 16 hours at 25 to 30° C. The resulting solids was filtered and washed with isopropyl acetate (20 ml). The resulting wet cake was dried under vacuum at 50 to 55° C. to produce 5.8 g of Lercanidipine hydrochloride Form Y.

### Example 5

### Purification of Form Y of Lercanidipine Hydrochloride

The Lercanidipine hydrochloride Form Y (4.0 g) prepared as per example 4 was dissolved in N,N-dimethyl acetamide (6 ml) at 60 to 65° C. under stirring. This was followed by the addition of isopropyl acetate (60.0 ml) under stirring. The resulting solution was cooled at 20 to 25° C. The resulting mass was further stirred for 15-17 hours at 20 to 25° C. The resulting solid was filtered and washed with isopropyl acetate (10 ml). The resulting wet cake was dried under vacuum at 50 to 55° C. to yield 2.5 g of pure Lercanidipine hydrochloride Form Y (HPLC Purity: 99.9%).

### Example 6

### Preparation of Amorphous Lercanidipine Hydrochloride

Lercanidipine hydrochloride (2 g) and water (100 ml) were heated under stirring for 150 minutes at 95-100° C. The resulting mass was cooled at 25-30° C. and then further cooled at 5-10° C. The reaction mixture was further stirred for 60 minutes at 5-10° C. The resulting yellow precipitated solid was filtered and washed with water. The resulting solid was dried at 60-65° C. to yield amorphous Lercanidipine hydrochloride (Yield=1.4 g, HPLC purity: 99.96%).

### Example 7

### Preparation of Amorphous Lercanidipine Hydrochloride

Lercanidipine hydrochloride Form Y (2 g) and water (100 ml) were heated under stirring for 150 minutes at 95-100° C. The resulting mass was cooled at 25-30° C. and then further cooled at 5-10° C. The reaction mixture was further stirred for 60 minutes at 5-10° C. The resulting yellow precipitated solid was filtered and washed with water. The resulting solid was dried at 60-65° C. to yield amorphous Lercanidipine hydrochloride (Yield=1.4 g, HPLC purity: 99.95%).

1. A process for the preparation of lercanidipine hydrochloride, optionally in the form of polymorph Form Y or optionally in amorphous form, which process comprises:
reaction of a compound of formula II: 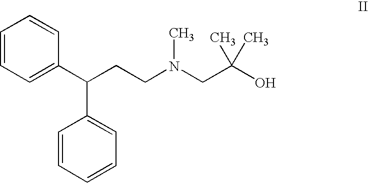
with a compound of formula III: 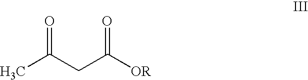
wherein R is an alkyl or aryl group to form a compound of formula I: 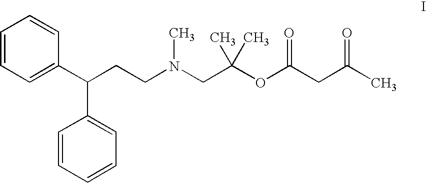
(ii) conversion of the compound of formula I to lercanidipine hydrochloride by reaction of the compound of formula I with 3-nitrobenzaldehyde to produce 1,1,N-trimethyl-N-(3,3-diphenylpropyl)-2-aminoethyl α-acetyl-3-nitrocinnamate followed by cyclization with methyl 3-aminocrotonate and reaction with a hydrochloride solution;
(iii) and thereafter optionally obtaining polymorph Form Y of lercanidipine hydrochloride by:
a) providing a solution of lercanidipine hydrochloride in an amide solvent;
b) adding an aliphatic ester solvent to the solution; and
c) recovering substantially pure lercanidipine hydrochloride Form Y from the solution;
(iv) and thereafter optionally obtaining amorphous lercanidipine hydrochloride by:
a) dissolving the lercanidipine hydrochloride in water;
b) heating the solution; and
c) recovering lercanidipine hydrochloride in amorphous form.

2. A process for the preparation of 1,1,N-trimethyl-N-(3,3-diphenylpropyl)-2-aminoethyl acetoacetate of formula I: 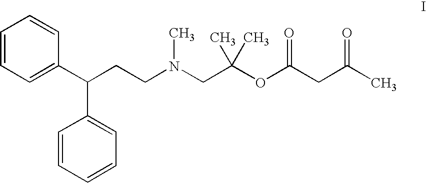
which comprises:
reacting 2,N-dimethyl-N-(3,3-diphenylpropyl)-1-amino-2-propanol of formula II: 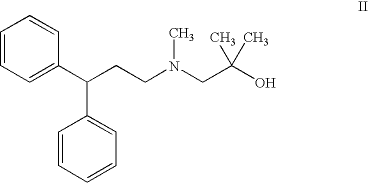
with a protected acetoacetic acid compound of formula III: 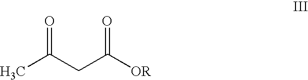
wherein R is an alkyl or an aryl group, in the presence of a metal catalyst in a suitable solvent to produce substantially pure 1,1,N-trimethyl-N-(3,3-diphenylpropyl)-2-aminoethyl acetoacetate of formula I.

3. The process of claim 2, wherein the metal catalyst is selected from the group consisting of Zn, Sc, Ti, V, Cr, Mn, Fe, Co, Ni and Cu.

4. The process of claim 3, wherein the metal catalyst is Zn.

5. The process of claim 2, wherein the solvent comprises non-polar solvents, aprotic solvents, alcohol solvents, and mixtures thereof.

6. The process of claim 5, wherein the solvent is selected from the group consisting of toluene, xylene, dimethylformamide, dimethylacetamide, dimethylsulfoxide, methanol, ethanol, n-propanol, isopropanol, n-butanol, isobutanol, tert-butanol, benzyl alcohol, benzyloxyethanol, phenoxyethanol, and mixtures thereof.

7. The process of claim 6, wherein the solvent is selected from the group consisting of toluene, xylene, dimethylformamide, and mixtures thereof.

8. The process of claim 2, wherein the protecting group R is selected from the group consisting of methyl, ethyl, n-propyl, isopropyl, n-butyl, t-butyl, pentyl, and benzyl groups.

9. The process of claim 8, wherein the protecting group R is methyl.

10. The process of claim 2, wherein the metal catalyst is used in a molar ratio of about 0.5 to 6 moles per 1 mole of 2,N-dimethyl-N-(3,3-diphenylpropyl)-1-amino-2-propanol.

11. The process of claim 10, wherein the metal catalyst is used in a molar ratio of about 2 to 4 moles per 1 mole of 2,N-dimethyl-N-(3,3-diphenylpropyl)-1-amino-2-propanol.

12. The process of claim 2, wherein the protected acetoacetic acid compound of formula III is used in a molar ratio of about 3 to 15 moles per 1 mole of 2,N-dimethyl-N-(3,3-diphenylpropyl)-1-amino-2-propanol.

13. The process of claim 12, wherein the protected acetoacetic acid compound of formula III is used in a molar ratio of about 8 to 11 moles per 1 mole of 2,N-dimethyl-N-(3,3-diphenylpropyl)-1-amino-2-propanol.

14. The process of claim 2, wherein the 1,1,N-trimethyl-N-(3,3-diphenylpropyl)-2-aminoethyl acetoacetate obtained has a purity of greater than about 99% as measured by HPLC.

15. (canceled)

16. A substantially pure 1,1,N-trimethyl-N-(3,3-diphenylpropyl)-2-aminoethyl acetoacetate.

17. The compound of claim 16, wherein the 1,1,N-trimethyl-N-(3,3-diphenylpropyl)-2-aminoethyl acetoacetate having a purity of greater than about 99% as measured by HPLC.

18-20. (canceled)

21. A crystalline lercanidipine hydrochloride Form Y characterized by a powder X-ray diffraction pattern having peaks at about 4.7 and 5.0+0.2 degrees 2-theta.

22. The crystalline form of claim 21, wherein the crystalline form is further characterized by at least one of the following: a powder XRD pattern having additional peaks at about 7.0, 18.9, 23.1 and 24.9+0.2 degrees 2-theta substantially as depicted in FIG. 1; a DSC thermogram having an endotherm peak at about 173° C. substantially as depicted in FIG. 2; a TGA thermogram substantially in accordance with FIG. 3; and/or an IR spectrum substantially in accordance with FIG. 4.

23. A process for the preparation of crystalline lercanidipine hydrochloride Form Y of claim 21, which comprises:
a) providing a solution of lercanidipine hydrochloride in an amide solvent;
b) adding an aliphatic ester solvent to the solution; and
c) recovering substantially pure lercanidipine hydrochloride Form Y from the solution.

24. The process of claim 23, wherein the amide solvent used in step-(a) is selected from the group consisting of N,N-dimethylacetamide, N,N-diethylacetamide, N,N-dimethylacetoacetamide, N,N-diethylacetoacetamide, formanilide, N-methyl formanilide, N,N-di-n-propyl acetamide, N,N-di-isopropyl acetamide, Di-n-butyl-acetamide, N,N-dimethyl-2,2-diphenyl acetamide, and mixtures thereof.

25. The process of claim 24, wherein the amide solvent is N,N-dimethylacetamide.

26. The process of claim 23, wherein the solution in step-(a) is prepared by dissolving any form of lercanidipine hydrochloride in a suitable amide solvent, or obtaining an existing solution from a previous processing step.

27. The process of claim 23, wherein the solution in step-(a) is prepared by admixing lercanidipine free base, hydrochloric acid and the amide solvent to obtain a mixture; and heating the mixture to obtain a lercanidipine hydrochloride solution.

28. The process of claim 23, wherein the aliphatic ester solvent used in step-(b) comprises ester groups having from about 2 to about 12 carbon atoms.

29. The process of claim 28, wherein the aliphatic ester solvent is selected from the group consisting of ethyl acetate, isopropyl acetate, and mixtures thereof.

30. A process for the preparation of amorphous lercanidipine hydrochloride, which comprises:
a) suspending lercanidipine hydrochloride in water;
b) heating the suspension; and
c) recovering lercanidipine hydrochloride in amorphous form.

31. The process of claim 30, wherein the suspension in step-(b) is heated to a temperature ranging from about 60° C. to about 100° C.

32. The process of claim 31, wherein the suspension is heated at 95 to 100° C. for a time period ranging from about 40 minutes to about 120 minutes.

33. A substantially pure lercanidipine hydrochloride Form Y.

34. (canceled)

35. The compound of claim 33, wherein the lercanidipine hydrochloride Form Y having a purity of greater than about 99.7% as measured by HPLC.

36. A pharmaceutical composition comprising lercanidipine hydrochloride Form Y of claim 21 and a pharmaceutically acceptable excipient.

37. The pharmaceutical composition of claim 36, wherein the pharmaceutical composition is selected from a solid dosage form and an oral suspension.

38. A pharmaceutical composition comprising lercanidipine hydrochloride Form Y crystalline particles, wherein 90 volume-% of the particles (D₉₀) have a size of less than 400 microns.

39. (canceled)

40. The pharmaceutical composition of claim 38, wherein the 90 volume-% of the particles (D₉₀) have a size of less than or equal to about 200 microns.

41. (canceled)

42. The pharmaceutical composition of claim 40, wherein the 90 volume-% of the particles (D₉₀) have a size of less than or equal to about 15 microns.
